(19) United States
(12) Patent Application Publication
(10) Pub. No.: US 2008/0293999 A1
(43) Pub. Date: Nov. 27, 2008

(54) Medical devices with portions having different rigidity

(75) Inventor: Izhar Halahmi, Hod HaSharon (IL)

Correspondence Address: Martin D. Moynihan;PRTSI, Inc., P.O. Box 16446, Arlington, VA, 22215, US

(73) Assignee: Vision - Sciences Inc., Orangeburg, NY (US)

(21) Appl. No.: 12/081,764

(22) Filed: Apr. 21, 2008

Related U.S. Application Data
(60) Provisional application No. 60/907,896, filed on Apr. 20, 2007.

(51) Int. Cl. A61B 1/00; B29C 65/06

(52) U.S. Cl. 600/101; 156/73.5

(57) ABSTRACT

A medical probe, including a rigid proximal portion formed from a blend having a 1% secant flexural modulus of at least 1 GPa including at least 5% by weight of Liquid Crystal Polymer (LCP) having a melting point not lower than 280° C.; a flexible distal portion having a shore A hardness lower than 90 A; and a weld joining the distal portion to the proximal portion.

### RELATED APPLICATIONS

The present application claims the benefit of U.S. Provisional Patent Application No. 60/907,896, filed on Apr. 20, 2007. The contents of the above-mentioned application are incorporated herein by reference.

### FIELD OF THE INVENTION

The present invention relates to thermoplastic blends and particularly to blends characterized by a high degree of rigidity and weldability with thermoplastic elastomers.

### BACKGROUND OF THE INVENTION

Endoscopes are used to view internal tissue of humans, and for many other tasks. In order to properly progress into the patient's body, the endoscope needs to be very narrow and have specific rigidity characteristics. In many cases it is desired to have a proximal rigid section, to allow pushing of the endoscope and a distal flexible section to allow bending and/or conforming to body tissue. Many ways of achieving variable flexibility along the length of an endoscope have been proposed. The following listing describes representative strategies for production of endoscopes with variable flexibility. The list does not purport to be exhaustive.

U.S. Pat. No. 4,690,175 to Ouchi et al., issued on Sep. 1, 1987, the disclosure of which is incorporated herein by reference, describes an endoscope having flexibility which varies in a stepwise manner along its length. The endoscope is formed of an inner core and a plurality of thermoplastic synthetic resin tube sections of different hardness bonded to the core at different locations along its length. This endoscope structure is relatively complex and has relatively thick walls. The multilayer structure is more difficult to manufacture and calls for expensive tooling.

U.S. Pat. No. 6,143,013 to Samson et al., issued on Nov. 7, 2000, the disclosure of which is incorporated herein by reference, describes an endoscope having a proximal section more rigid than the distal section. Samson describes attaching the proximal and distal segments using a conical or scarf joint. This method of attachment involves having a weak point along the length of the endoscope.

U.S. Pat. No. 5,961,511 to Mortier et al., issued on Oct. 5, 1999, the disclosure of which is incorporated herein by reference, describes a catheter having a proximal portion which is rigid relative to its distal portion. In one embodiment therein, the proximal portion has an additional stiff jacketing layer formed of a material such as polyimide, PVC, polyethylene or PET. In another embodiment thereof, the proximal and distal portions are formed from separate elements which are joined by an adhesive or fastener. Such joining methods are generally not sufficiently reliable and involve use of an additional agent which makes the manufacture more complex. In still another embodiment, one layer is more rigid in a proximal portion than in a distal portion and at a transition point the layer is formed as a combination of the materials of the proximal and distal portions. The proximal portion is formed by extrusion of a stiffening layer, such as polyimide, polyamide or polyurethane. The distal portion includes a braided layer formed of strands of LCP.

The above medical tubes require complex production methods and have relatively thick walls.

Liquid crystal polymers (LCPs) are wholly or partially aromatic polyester polymers characterized by high performance properties, including high modulus of elasticity (rigidity) and strength and chemical resistance. LCPs comprise densely packed fibrous-like polymer “chains” that provide self-reinforcement almost to their melting point. LCPs can be melt processed on commercially available equipment at fast speeds. LCPs are also capable of forming regions of ordered structure while in the liquid phase, although less ordered than a regular solid crystal.

U.S. Pat. No. 7,026,026 to Ferrera et al., issued on Apr. 11, 2006, the disclosure of which is incorporated herein by reference, describes catheter balloons formed from LCP, a crystallizable thermoplastic polymer, such as PET and a compatibilizer.

U.S. Pat. No. 7,101,597 to Wang et al., issued on Sep. 5, 2006, the disclosure of which is incorporated herein by reference, describes a medical device including a polymeric material formed as a melt blend product of at least two different thermoplastic polymers, one of the thermoplastic polymers being an LCP having a melting point of less than 250° C. The portion of the device made from the melt blend may be a catheter body segment or a balloon for a catheter. The LCP blends also include a non-LCP base polymer having a melting point in the range of about 140 to about 265° C. Wang describes use of polymeric material including LCP to improve length stability of balloons. The use of LCP with a low melting point limits the rigidity achievable.

U.S. Pat. No. 5,441,489 to Utsumi et al., issued on Aug. 15, 1995, the disclosure of which is incorporated herein by reference, describes a catheter having a rigid proximal portion and a short flexible distal portion. The portions are mixed and fusion connected or successively extruded. Optionally, an intermediate portion is provided or formed by the extrusion process. The proximal and distal portions have the same inner and outer diameters. In one embodiment, the distal portion comprises polyurethane, while the proximal portion comprises a blend of 80% LCP and 20% polyurethane. The LCPs described by Utsumi are characterized by a melting temperature below 280° C., preferably at 150-250° C., due to the poor thermal stability of the polyurethane. It is noted that polyurethane cannot withstand temperatures above 250° C. and practically not even above 220° C. LCPs with melting points of below 250° C. are less stiff and may not provide a sufficient stiffness.

### SUMMARY OF THE INVENTION

An aspect of some embodiments of the present invention relates to an elongate tube or other probe of a medical instrument (e.g., endoscope, catheter) having at least two portions, which have substantially different properties but are welded to each other. A proximal portion is formed from a rigid polymeric blend, comprising a substantial amount of a Liquid Crystal Polymer (LCP) characterized by a melting temperature above 270° C. or even above 280° C. A more distal portion, possibly a distal portion of the elongate tube, is formed of a flexible (e.g., elastomeric) thermoplastic polymer, such as a polyurethane. Using weldable materials for the different portions allows achieving a strong bond between the portions, without forgoing the desired properties of either of the proximal and distal portions.

The rigid polymeric blend optionally includes an engineering thermoplastic resin, which is thermally stable at a temperature above the melting point of the LCP in the blend and is relatively rigid at room and/or human body temperature. The rigid polymeric blend can optionally be molded or extruded into thin walls. Alternatively to a single thermoplastic resin, the rigid blend includes a plurality of thermoplastic resins, at least a first one of which is relatively rigid and at least a second one of which is more weldable to the flexible thermoplastic.

In some embodiments of the invention, a compatibilizer is added to the rigid blend in order to provide stable dispersion of the LCP and the engineering thermoplastic resin in the blend, during manufacture, and/or to provide the blend with suitable mechanical and physical properties, for medical tubes.

Alternatively or additionally to an engineering thermoplastic resin the rigid blend comprises a softer material, which has good welding properties.

The flexible thermoplastic optionally has a shore hardness lower than about 90 A or even lower than 75 A, to allow sufficient flexibility for maneuvering through narrow body passages within a patient, such as the large intestine or the small intestine. In some embodiments of the invention, the thermoplastic has a shore hardness lower than 60 A or even lower than 40 A.

In some embodiments of the invention, the tensile modulus of elasticity of the proximal portion of the elongate tube is at least 1.2 or even 1.5 GPa (Giga Pascal), to provide sufficient stiffness for pushability within the patient.

There is therefore provided in accordance with an exemplary embodiment of the invention, a medical probe including a rigid proximal portion formed from a blend having a 1% secant flexural modulus of at least 1 GPa including at least 5% by weight of Liquid Crystal Polymer (LCP) having a melting point not lower than 280° C., a flexible distal portion having a shore A hardness lower than 90 A and a weld joining the distal portion to the proximal portion.

Optionally, the blend has a 1% secant flexural modulus of at least 1.2 GPa, possibly at least 1.5 GPa or even at least 3 GPa.

Optionally, the LCP is a polyester. Alternatively or additionally, the rigid blend comprises a polymeric substance in addition to the LCP. Optionally, the polymeric substance forms at least 30% of the blend. In some embodiments of the invention, the polymeric substance comprises an engineering resin. Optionally, the engineering resin has a modulus in the range of 1.0-2.0 GPa. Alternatively, the engineering resin has a modulus above 2.0 GPa.

Optionally, the engineering resin is selected from the group consisting of a polysulfone, a polyether ether ketone (PEEK), a polyphenylene sulfide (PPS), a polyphenylene ether (PPE), a polyphthalamide (PPA) and a polyimide (PI). Optionally, the engineering resin comprises at least one of a polyester, a polyamide, a polyester copolymer and a polyamide copolymer.

Optionally, the polyamide is selected from the group consisting of polyamide 6, polyamide 66, polyamide 11, polyamide 12, polyamide 46, polyamide 6T, Polyphthalamide (PPA), blends and copolymers thereof.

Alternatively or additionally, the polyester is selected from the group consisting of polyethylene terephthalate (PET), polybutylene terephthalate (PBT), glycol-modified polyethylene terephthalate (PETG), copolyesters and Poly ethylene naphthalate (PEN).

Optionally, the polymeric substance comprises a polyether block amide (PEBA).

Optionally, the probe includes a compatibilizer forming at least 1% of the blend or even at least 10% of the blend. In some embodiments of the invention, the compatibilizer comprises a substantially greater portion of the blend than required for stable and repeatable coexistence of the LCP and the polymeric substance.

Optionally, the rigid blend further comprises at least 5% of a polymer or oligomer characterized by an average molecular weight not exceeding 20,000 Daltons.

Optionally, the polymer or oligomer has a melting point not exceeding 250° C. Possibly, the polymer or oligomer has a softening point not exceeding 250° C. Optionally, the LCP forms at least 20% of the blend.

Optionally, the blend forming the rigid proximal portion is weldable to thermoplastic polyurethane (TPU).

Optionally, the flexible distal portion comprises a TPU, selected from polyester-urethane, polyether-urethane, polycarbonate-urethane and silicone-urethane. Optionally, the flexible distal portion has a shore A hardness lower than 75. Optionally, the rigid proximal portion has a wall having a thickness of less than 0.2 millimeters.

There is further provided in accordance with an exemplary embodiment of the invention, a manufacturing method, including providing a first part formed from a rigid polymeric blend, comprising at least 5% LCP having a melting point not lower than 280° C.; and a polymeric substance. The blend is characterized by a 1% secant flexural modulus of at least 1.2 GPa. The method further includes further providing a second part formed from a flexible polymeric material and joining the first part and the second part by welding.

Optionally, the flexible polymeric material comprises polyurethane. In some embodiments of the invention, the welding is performed by application of heat and/or application of a solvent. Optionally, each of the first and second parts are independently provided in the form of a rod or a tube. Optionally, the first part has a thickness of less than 0.2 millimeters. Optionally, the method includes adjusting a diameter of an end of at least one the first part and the second part so that the adjusted end fits over an end of the other part. Optionally, the adjusting includes at least one process selected from the group consisting of thermoforming, stretching and solvent swelling. In some embodiments of the invention, the flexible polymeric material has a shore hardness lower than 90 A at 20-25° Celsius.

### BRIEF DESCRIPTION OF THE DRAWINGS

Exemplary non-limiting embodiments of the invention will be described with reference to the following description of the embodiments, in conjunction with the figures. Identical structures, elements or parts which appear in more than one figure are preferably labeled with a same or similar number in all the figures in which they appear, and in which:

FIG. 1 is a schematic illustration of an endoscope system, in accordance with an exemplary embodiment of the present invention; and

FIG. 2 is a flowchart of acts performed in production of the endoscope system of FIG. 1, in accordance with an exemplary embodiment of the invention.

### DETAILED DESCRIPTION OF EXEMPLARY EMBODIMENTS

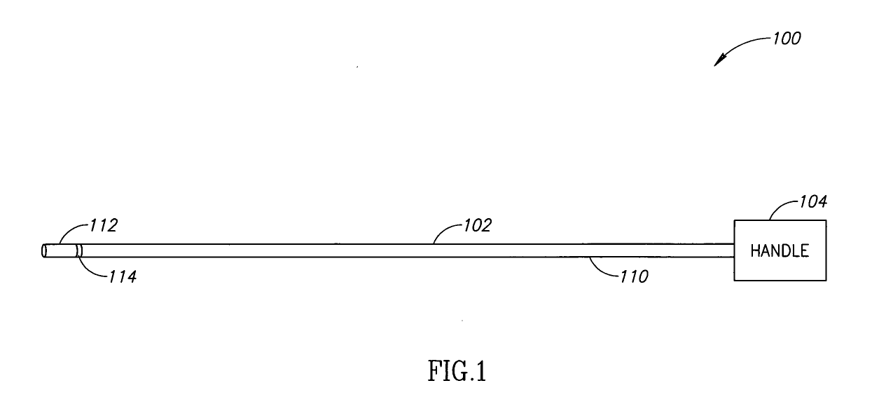

FIG. 1 is a schematic illustration of an endoscope system 100, in accordance with an exemplary embodiment of the present invention. System 100 optionally includes an elongate insertion tube 102 and a handle 104. Endoscope system 100 may be of substantially any type and may be used for substantially any endoscopic procedure and the specific details (e.g., size, shape, elements included) of insertion tube 102, handle 104 and the other parts of system 100 are selected according to the intended uses of system 100.

Insertion tube 102 comprises a proximal portion 110 and a distal portion 112 formed of materials of substantially different characteristics, such that proximal portion 110 is much more rigid than distal portion 112. Portions 110 and 112 are welded together at an intermediate portion 114. Intermediate portion 114 may be relatively long, for example of a length of at least 5 or even 10 millimeters, or may be very short, for example less than 2 or even less than 1 millimeter.

The use of the compositions discussed hereinbelow, allows production of very thin endoscope tubes. For example, tube 102 may have a thickness (i.e., the difference between the inner and outer diameter) of less than 0.5 millimeters, less than 0.2 millimeters or even less than 0.1 millimeters. It is noted, however, that the use of these compositions is advantageous also in other medical tubes, having thicker walls and even in medical tubes having a metal reinforcement running therethrough. Tube 102 may have substantially any suitable inner diameter, for example between 1.5-15 millimeters, although smaller or larger diameters may be used. In some embodiments of the invention, tube 102 has a substantially same thickness over both of portions 110 and 112. Alternatively, tube 102 has different thickness in the different portions, optionally being thicker in distal portion 112.

In some exemplary embodiments of the invention, the tube is formed such that it has shape memory such that it attempts to return to a preferred state, for example using heat shrink methods. The blend from which the tube is produced, at least in these exemplary embodiments, is suitable for heat shrink handling.

Production Method

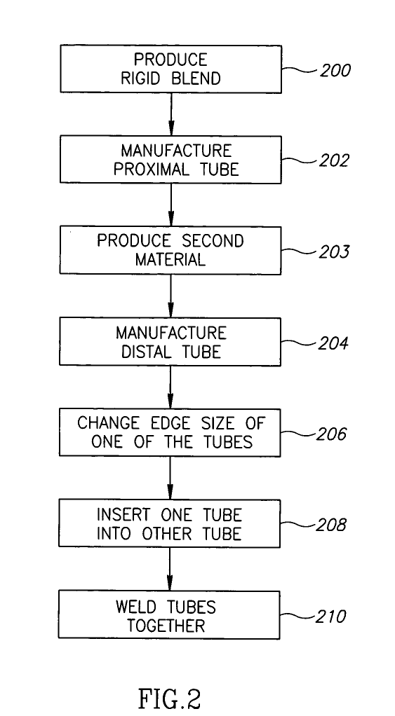

FIG. 2 is a flowchart of acts performed in producing insertion tube 102, in accordance with an exemplary embodiment of the invention. A first thermoplastic blend, having rigid characteristics provided by a substantial amount of LCP is produced (200) and a tube for proximal portion 110 is manufactured (202) from the first thermoplastic blend. A second thermoplastic material, having flexible characteristics, is produced (203) and a tube, tip or molded cap for distal portion 112 is manufactured (204) from the second material. A size of an edge of one of the tube portions is optionally changed (206), so that one of the tubes can be fit into the other tube, i.e., the outer diameter on the edge of one tube is slightly smaller than the inner diameter of the second tube. The tube with the smaller edge is optionally inserted (208) into the other tube, and the tubes are then welded (210) together.

Referring in more detail to the manufacture of the tubes for proximal portion 110 and distal portion 112, in some embodiments of the invention, the tubes are manufactured by extrusion, for example when a tube with relatively thick walls is desired. Alternatively, thermoforming or blow molding is used in manufacturing one or more of the tubes for portions 110 and 112, for example when a tube having thin walls (e.g., less than 0.5 millimeters) and/or a complicated structure (e.g., being corrugated, including a plurality of lumens, having an irregular cross section and/or being reinforced) is required. Further alternatively or additionally, any other polymer product shaping method known in the art is used in manufacturing the tubes, such as injection molding, or dip molding. The dip molding may optionally be performed from a melt or a solution. Dip molding is optionally used when the blend from which the tube is produced has a very high melting point and/or is relatively heat sensitive. In some embodiments of the invention, the same production method is used for both the proximal and distal portions. Alternatively, different production methods are used for proximal portion 110 and distal portion 112.

Optionally, only one of the tubes is treated to change (206) the size of its edge. Alternatively, the sizes of edges of both the tubes may be adjusted to fit one in the other. The size adjustment is optionally performed using one or more of thermoforming, stretching and/or solvent swelling. Further alternatively, the tubes are initially produced with slightly different sizes, at least at their edges, so that they fit into each other. The fitting may be with or without interference, as appropriate.

In some embodiments of the invention, the tube for proximal portion 202 is stretched during production and then cooled in a quick process, such that in use, upon applying heat, the tube shrinks. Alternatively or additionally, the tube is designed to shrink in response to one or more other physical triggers, such as actinic irradiation, a magnetic or electric field and/or exposure to a chemical agent.

Welding

Referring in detail to inserting (208) one of the tubes into the other, in some embodiments of the invention the tube is inserted only slightly, fitting in less than 2 millimeters, less than 1 millimeter or even less than 0.5 millimeters of the axial length of the other tube. In some embodiments of the invention, the tube is inserted to an extent as small as 0.05 millimeters or even less. Alternatively, the tube is inserted to a larger extent of at least 2, at least 5 or even at least 8 millimeters. In some embodiments of the invention, the tube is inserted for an extent of 10 millimeters or even more. The extent of insertion optionally determines the length of welding portion 114. Optionally, the tube of distal portion 112 is inserted into the tube of proximal portion 110, although the opposite may also be performed.

For purposes of this specification and the accompanying claims, the term “weldable” indicates an ability of two polymeric materials to form a durable, permanent joint as a result of application of energy and/or solvents.

The result of the welding is optionally a zone, usually 1-500 microns thick, where molecular blending takes place between the two different polymers.

The welding (210) of the tubes is optionally performed using any suitable method known in the art, such as hot bar welding, ultrasonic welding, laser welding, hot air welding, radiofrequency (RF) welding or solvent bonding. Solvents suitable for use in welding polymeric materials include, but are not limited to, ketones, amides, cresols, esters, ethers, phenols, alcohols, chlorinated hydrocarbons, lactones, lactams, heterocyclic amines and combinations thereof.

Optionally, the second material is weldable to the first thermoplastic blend via an elastomeric phase of the second material which welds to an engineering thermoplastic phase of the first blend. Since the two phases have lower melting temperatures than the LCP, and optionally lower than the melting temperature of at least a portion of the engineering thermoplastic phase, the welding process is enabled at temperatures below the melting point of the rigid sector of the welded tubes.

Material of First Blend

The first thermoplastic blend is optionally chosen to have a strength of at least 10 MegaPascal (Mpa) or even at least 30 Mpa. Optionally, the first thermoplastic blend has a high chemical stability, so that it does not delaminate or separate after the blend is solidified. The first thermoplastic blend optionally has a tensile modulus of elasticity of at least 1.2 or even 1.5 GPa (Giga Pascal). In some embodiments of the invention, the first thermoplastic blend has a tensile modulus of elasticity of at least 2 GPa, 3.5 GPa and even at least 4 GPa. Such rigidity can allow even very thin walls to be sufficiently rigid so that insertion tube 102 can be introduced into a body cavity, without risk of collapsing. In the present application, the values of modulus are defined for 1% secant flexural modulus, according to ASTM D790.

In some embodiments of the invention, the first thermoplastic blend is suitable for heat shrink processing, due to its formation from LCP and an engineering resin which have complimentary characteristics.

The first thermoplastic blend is optionally processable by at least one of extrusion, blow molding, thermoforming and injection molding. Optionally, the first blend is produced by melt kneading all its components together in a mixer or an extruder.

The first blend is optionally formed from a substantial amount of LCP blended with an engineering thermoplastic resin. In some embodiments of the invention, the first blend further comprises a compatibilizer, which provides stable dispersion of the LCP and the thermoplastic resin in the blend. A compatibilizer is optionally used when the first blend includes PEBA and/or polyamide. In contrast, when the engineering blend comprises PET and/or copolyester, a compatibilizer is optionally not included in the first blend. Other materials in the first blend, for example those discussed hereinbelow, are optionally included in relatively small amounts, so that they do not adversely affect the properties of the blend.

LCP

The first thermoplastic blend optionally includes at least 5% or even at least 10% LCP of a high melting point. Furthermore, for endoscopes requiring high rigidity, a percentage of LCP in the blend could be at least 15% or even at least 20% or 30%. It is noted however that in some cases more than 40% or even more than 50% of the blend comprises LCP. In some embodiments of the invention, the amount of LCP is not too high, so that insertion tube 102 can be manipulated to a required extent. Accordingly, in some embodiments of the invention, less than 30% or even less than 20% of the first blend is LCP. In other embodiments, only a small amount of LCP is used, for example less than 15% or even less than 10%. It is noted that although specific numbers were given, the percentage of LCP included in the first blend may be in a very wide range of 5-60% or even more or less.

The exact amount of LCP used is optionally determined according to a required rigidity of insertion tube 102. In some embodiments of the invention, the amount of LCP used is a function of the thickness of the walls of proximal portion 110. Optionally, a higher percentage of LCP is used when thinner walls are desired, in order to provide the additional stiffness required by the thin walls. Alternatively or additionally, the percentage of LCP included in the first blend is a function of the desired maneuverability of the proximal portion 110, which is selected according to the body organ for which endoscope system 100 is designed.

The LCP used is optionally one with a high melting temperature, for example higher than 260° C. or even more than 280° C. or 300° C., as such LCPs enjoy a high stiffness. In some embodiments of the invention, the LCP used in the first blend has a stiffness greater than 2.5 GPA, greater than 3.5 GPa, greater than 4 GPa or even greater than 5 GPa. In an exemplary embodiment of the invention, the LCP has a stiffness greater than 3 GPA.

In some embodiments of the invention, only a single type of LCP is used in the first blend, for simplicity. Alternatively, the first blend includes a plurality of different LCPs. Exemplary LCPs which may be included in the first blend comprise Vectra™ manufactured by Ticona (http://www.ticona.com/), Xydar™ manufactured by Solvay (http://www.solvayadvancedpolymers.com/) and Siveras™ manufactured by Toray (http://www.toray.com/).

Engineering Resin

Optionally, the majority of the remaining portion of the first polymeric composition comprises an engineering thermoplastic resin, which is weldable to the second material which has flexible characteristics and remains chemically stable, and is workable at, a temperature above 250° C., more specifically a temperature above the melting point of the LCP included in the first blend. It is noted that the term resin is used herein in its broad meaning as referring generally to industrially useful polymers. Nonetheless, in some embodiments of the invention, the engineering resin has the properties of resin in its more narrow meaning, e.g., stickiness and/or hardening in air.

In some embodiments of the invention, the engineering thermoplastic resin is relatively rigid at room temperature, having on its own a tensile modulus of elasticity of at least 0.8, 1.2 GPa or even at least 2 GPa, so that it does not counteract the rigidity of the LCP. In an exemplary embodiment, the engineering thermoplastic resin has a tensile modulus of elasticity of at least 3 GPa. Optionally, the engineering resin has a low melting temperature, or at least workable temperature, relative to the LCP, optionally having a melting temperature below 250° C., or even below 220° C. In some embodiments of the invention, the engineering thermoplastic resin used in the blend has a melting temperature below 200° C. or even below 190° C. For example, when a high level of toughness and/or weldability to polyurethane or the like is desired, tough and/or weldable materials, such as PA12, PA 11 or PEBA, which generally have a low melting temperature, are used.

Exemplary materials which may serve as the engineering thermoplastic resin comprise semi-flexible (i.e., having a modulus in the range of 1.0-2.0 GPa) engineering thermoplastics, for example one or more of:

1) Polyamide resin, such as polyamide 11, polyamide 12, polyamide 46 and polyamide 6T

2) Co-polyester which are highly weldable to flexible thermoplastics.

Alternatively or additionally, one or more high modulus (i.e., having a modulus above 2.0 GPa) engineering thermoplastics is used in the first blend as the engineering resin, such as one or more of:

3) Polyester resins, such as polyethylene terephthalate (PET), polybutylene terephthalate (PBT), glycol-modified polyethylene terephthalate (PETG), rigid copolyesters, Poly ethylene naphthalate (PEN) and Polycarbonate.

4) Polyamide 6, polyamide 66, polyamide 6T, polyamide 9T, polyamide 46 and semi-aromatic polyamide

5) Polyether ether ketone (PEEK)

6) Polyphthalamide (PPA) blends

7) Polyimide (PI)

8) Polyamide-imide

9) Polyetherimide (PEI), polyphenylenesulfide (PPS), polyphenylene-ether (PPE)

It is noted that some copolymers of the above materials may also be used.

The percentages of the LCP and engineering resin in the first blend optionally depend on the specific engineering resin used. Optionally, when a high modulus engineering resin is used, the LCP percentage is in the range 5-20%, while when a low modulus engineering resin is used, the percentage of LCP is the range 15-40%. The engineering resin optionally forms at least 30% or even at least 50% of the first blend.

While the engineering thermoplastic resin may be formed of a single thermoplastic material, in some embodiments of the invention, the resin includes a plurality of materials, which are melt kneaded together with the LCP or in advance.

Softer Polymers

Alternatively to using an engineering resin, the first blend may comprise a substantial amount of a softer polymer, which remains stable at relatively high temperatures, such as polyether block amides (PEBA). Such a soft polymer, may be used, for example, to allow for easier weldability with soft materials. When such softer polymers are used, the LCP content is optionally at least 20%, at least 30% or even at least 40% in order that the first blend be sufficiently rigid.

Compatibilizer

In some embodiments of the invention, materials which are compatible with LCP, such as PET, PBT, PEN, Polycarbonate, copolyesters and polyester-amides, are used as the thermoplastic engineering resin, in order to avoid the need for a compatibilizer. Alternatively, other materials which have preferred characteristics are used, although they require use of a compatibilizer. The compatibilizer is optionally used when the LCP and engineering resin are incompatible with each other. Alternatively or additionally, a compatibilizer is included when a blend of the LCP and the engineering thermoplastic resin does not have sufficient impact strength, does not have sufficient fatigue resistance, tends to melt fracturing and/or phase separation during extrusion and/or molding, does not provide repeatable welding with flexible polymers, does not provide sufficient resistance against sterilization processes, does not have sufficient tear resistance and/or does not have sufficient toughness (resistance to breakage).

The compatibilizer optionally includes an agent which is adapted to interact with both the LCP and the engineering resin, so as to form a stable blend. A stable blend is characterized by predictable and repeatable morphology of dispersed phase in continuous phase, and by mechanical, physical, rheological and chemical properties, which make the blend suitable for a designated application. The compatibilizer optionally lowers the enthalpy of mixing, encapsulating dispersed phase and blocking polar groups in said phases.

The compatibilizer may be provided in substantially any form, including powder, flakes, granules, pellets, liquid or solution.

In some embodiments of the invention, the compatibilizer forms more than 5% or even more than 10% of the first blend. The compatibilizer, however, optionally comprises less than 30% or even less than 15% of the first blend. In some embodiments of the invention, the compatibilizer forms less than 5%, less than 2% or even less than 1% of the first blend.

Possible materials which may be used as the compatibilizer include Maleic anhydride grafted polyethylene, Maleic anhydride grafted ethylene-acrylic ester co-polymers or terpolymers, Maleic anhydride grafted propylene homo-polymers and copolymers, Maleic anhydride grafted ethylene-alpha olefin polymers, Maleic anhydride grafted ethylene-propylene rubber, Glycidyl Methacrylate (GMA) grafted polyethylene, Glycidyl Methacrylate (GMA) grafted ethylene-acrylic ester co-polymer or terpolymer, Glycidyl Methacrylate (GMA) grafted propylene homo-polymers and copolymers, Glycidyl Methacrylate (GMA) grafted ethylene-alpha olefin polymers, Glycidyl Methacrylate (GMA) grafted ethylene-propylene rubber, acrylic or methacrylic acid grafted ethylene copolymers and terpolymers, acrylic and methacrylic acid ionomer and combinations thereof.

Examples of commercially available functionalized polymers, adapted to compatibilize the first blend as external compatibilizer according to some embodiments of the present invention are Lotader manufactured by Arkema, Bondyram manufactured by Polyram, Polybond manufactured by Crompton, Integrate manufactured by Equistar, Yparex manufactured by DSM, Primacor and Amplify manufactured by DOW, Epolene manufactured by Eastman, Escor and Optema and Exxelor manufactured by Exxon Mobil, Fusabond and Bynel and Elvaloy and Surlyn manufactured by Dupont, A-C modified polyolefins manufactured by Honeywell, Modic-AP manufactured by Mitsubishi, Admer manufactured by Mitsui, Modiper manufactured by NOF, and Igetabond manufactured by Sumitomo, styrene maleic anhydride copolymers and terpolymers, such as SMA resins manufactured by Sartomer, UMG AXS manufactured by UMG and Synthacryl manufactured by UCB resins.

In some embodiments of the invention, the first blend includes a minimal amount of compatibilizer required to allow for the coexistence of the LCP and the engineering resin. Alternatively, the amount of compatibilizer is larger than required for the coexistence of the LCP and the engineering resin. The excess compatibilizer may aid in the welding of the tube formed from the first blend, with the second tube.

Possibly, a first compatibilizer is used for the coexistence of the LCP and the engineering resin, while a second, different, compatibilizer is used to aid in the welding. The second compatibilizer may include, for example, anhydride, oxirane or carboxyl.

Soft Material

In some embodiments of the invention, the first blend additionally comprises a low molecular weight polymer that is characterized by higher fluidity relative to the LCP and engineering resin in the blend, so that the blend has higher diffusion rate during the welding stage. In an exemplary embodiment, the low molecular weight polymer has a molecular weight lower than 20,000 Daltons (D), 18,000 Daltons or even lower than 10,000 Daltons. Optionally, the low molecular weight polymer has a melting point lower than 250° C., 200° C. or even below 180° C. Optionally, the low molecular weight polymer forms at least 5% or even at least 10% of the first blend.

Examples for said low molecular weight polymer, are

(1) low melting point polyesters such as Crylcoat and Syntacryl manufactured by UCB, Fine-clad resins by Reichold, CAPA manufactured by Solvay, Baycoll manufactured by Bayer;

(2) liquid rubbers such as LIR liquid rubber by Kuraray;

(3) styrene-maleic anhydride copolymers and terpolymers such as SMA resins manufactured by Sartomer;

(4) acrylic and methacrylic acid copolymers and terpolymers such as Setalux and Setyrene manufactured by Akzo Nobel;

(5) polyamides such as Uni-Rez manufactured by Arizona Chemical; and

(6) polyester-urethane and polyether-urethane such as Bayhydrol manufactured by Bayer.

Additional Materials

The first blend may additionally include other materials which may add to the strength, rigidity and/or workability of the blend. Although it is advantageous that these materials remain stable at temperatures above 250° C., in some embodiments, they may be included in the first blend in small amounts (e.g., less than 1-2%), even if they cannot endure temperatures above 250° C.

In some embodiments of the invention, the first blend additionally includes a mineral filler, for example in the form of a fine powder, which adds to the stiffness, heat conductivity, crystallization rate, strength and/or contrast in X-ray imaging of the resulting tube. The mineral filler optionally includes less than 5% or even less than 3% of the entire blend, although in some cases the mineral filler may be more than 5% of the blend. Alternatively or additionally, the first blend includes one or more processing aids. Optionally, the processing aids include less than 5% or even less than 2% of the first blend.

Other materials which may be included in the first blend are plasticizers, release agents, heat stabilizers, anti-oxidants and colorants, including dyes and pigments. In some embodiments of the invention, an oil is added to the first blend, in order to provide for a smoother introduction of the endoscope into the patient. Germicides, anti-inflammatory and/or antibiotic drugs may also be included in the first blend, in small amounts.

Production Method

In producing (200) the first thermoplastic blend, the components of the blend are optionally placed together in an extruder, preferably a multi-screw extruder, such as a co-rotating twin screw extruder, in which they are melt kneaded together. The extruder distributes the polymers sufficiently so that they break into small enough pieces and form a homogeneous blend without segregation and/or formation of a layer structure.

The blending in the extruder is optionally performed at a temperature at which all the components melt, but which is not high enough to deteriorate any of the elements. In some embodiments of the invention, the blending is performed at a temperature above 230° C., above 250° C., or even above 280° C. In some embodiments of the invention, the blending is performed at a temperature above 300° C. or even above 330° C. Optionally, however, the blending is not performed at too high a temperature, which may damage the materials in the blend and change their properties. Therefore, in some embodiments of the invention, the blending is performed at a temperature below 300° C., below 270° C. or even below 250° C.

In some embodiments of the invention in which a compatibilizer is used, the compatibilizer is mixed with the LCP and engineering thermoplastic resin in a main hopper of the extruder or mixer. Alternatively, the compatibilizer is introduced into the extruder from a different port than used for the LCP and/or the engineering resin.

Material of Portion 112

Flexible distal portion 112 is formed from a second thermoplastic material which includes, in some embodiments of the invention, a flexible polymer or polymeric material having a shore hardness lower than 90 A or even lower than 75 A and is weldable to the first blend. In some embodiments of the invention, the flexible portion has a shore hardness lower than 60 A or even lower than 40 A.

Optionally, the second material is chemically stable and manufacturable into a tube or tip by extrusion, blow molding, dip molding, compression molding and/or injection molding.

The second material optionally includes a flexible (e.g., elastomeric) material, such as polyurethane. Examples of polyurethanes which may be used according to the present invention are Texin and Desmopan manufactured by Bayer, Estane manufactured by Noveon, Pellethane manufactured by Dow and Irogran manufactured by Huntsman.

Other materials which may be used as the second material include polyether block amides (PEBA), such as PEBAX manufactured by Arkema. Alternatively or additionally, the second material comprises a polyester or co-polyester, including copolymers and terpolymers thereof, for example Styrene block copolymers such as block copolymer of polystyrene blocks and ethylene-butylene blocks (SEBS) and block copolymer of polystyrene blocks and polyisoprene blocks (SIS), such as Kraton manufactured by Kraton polymers and Pabalon manufactured by Mitsubishi Chemical corp. Other polymers which may be used in the flexible material include polyolefin including modified polymers thereof, ethylene copolymers and terpolymers, and polyamides including copolymers and terpolymers thereof. An exemplary useful elastomeric polyester is Primalloy manufactured by Mitsubishi Chemical corp. Examples of useful elastomeric ethylene copolymers are Lotryl manufactured by Arkema, Elvaloy and Surlyn manufactured by Dupont and Acryft manufactured by Sumitomo chemicals.

Alternatively, the second material comprises a blend formed of any of the above listed flexible materials with an engineering thermoplastic resin, such as PET, polyamide, polycarbonate or polyester. In some embodiments of the invention, the engineering thermoplastic resin includes less than 30%, less than 20% or even less than 10% of the second material. Alternatively or additionally, the engineering thermoplastic resin includes more than 20%, 30% or even more than 40% of the second material. In some embodiments of the invention, the second material comprises a compatibilizer which allows the coexistence of the flexible material with the engineering thermoplastic resin.

The second polymeric composition may further comprise a plasticizer, oils, rubber liquid, pigments, dyes and/or processing aids. Exemplary plasticizers which may be used in the second material include phthalates, Phosphates, mono-, di- or poly-carboxylic esters, epoxidized oils and aryl sulfonamides.

The second blend is optionally produced using any suitable method known in the art, for example any of the methods described above for the first blend, although generally, lower temperatures are used, according to what is needed for the second material. In some embodiments of the invention, the second material is produced using melt kneading in an extruder, optionally in a co-rotating twin screw extruder, at a temperature range of between 180 to 250° C.

### Specific Examples

In an exemplary embodiment of the invention, the first blend comprises by weight, 37% LCP, 48% PEBA and 15% of a GMA grafted ethylene-acrylic ester copolymer compatibilizer. The flexible tube in this embodiment is optionally formed of thermoplastic polyurethane having shore hardness in the range of 60 A to 75 A. In this example, the LCP comprises the polymer Vectra™ manufactured by Ticona, the PEBA is PEBAX™ manufactured by Arkema, and the GMA grafted ethylene-acrylic ester copolymer is Lotader™ AX9800 manufactured by Arkema.

In manufacture, the LCP, PEBA and compatibilizer are blended (e.g., melt kneaded) together in a dry procedure. Optionally, in blending, a heat stabilizer Irgafos™ PEP-Q manufactured by Ciba in an amount of about 0.25% of the blended polymers total weight is added. The mixture is fed into a co-rotating twin screw extruder, having a length to diameter ration (L/D) of 40, equipped with one atmospheric vent aperture. The screw speed is optionally 200 RPM and barrel temperature is set to 290° C. The blend is optionally extruded as strands, cut to pellets and dried at 60° C.

In extrusion of a catheter, the pellets are optionally fed into a single screw extruder and extruded to a tube having an outer diameter of 5 mm and a wall thickness of 0.5 mm. The extruded tube is optionally further extended immediately after extrusion so as to reduce the wall thickness to 0.2 mm. The resultant tube is optionally heat bonded at a temperature of between 250-300° C. to a polyurathane tube, such as a tube manufactured from PELLETHANE™ 2363-90AE, manufactured by DOW.

### CONCLUSION

The above described endoscope may comprise substantially any type of endoscope, including a colonscope or laparoscope. Exemplary body cavities explored with the endoscope include, for example, the intestines, stomach, esophagus and urethra.

Alternatively or additionally to be used in producing an endoscope, the above described structure may be used in producing other medical tubes, such as catheters and delivery channels and/or protective sheaths for medical devices. The medical tubes are preferably produced in a sterilized environment and provided sealed in a sterilized kit.

It is noted that the above described principles may be used in producing an endoscope or other medical probe having more than two portions, for example three portions: LCP based, rigid plastic based and flexible. It is further noted that the LCP based portion does not necessarily extend up to the most proximal portion of the probe, but rather may include an intermediate portion which is referred to as proximal since it is proximal to the distal portion with which it is welded.

It will be appreciated that the above-described methods may be varied in many ways, including, changing materials, sizes and shapes. It should also be appreciated that the above described description of methods and apparatus are to be interpreted as including apparatus for carrying out the methods, and methods of using the apparatus. The percentages listed in the above description all refer to percentages by weight.

The present invention has been described using non-limiting detailed descriptions of embodiments thereof that are provided by way of example and are not intended to limit the scope of the invention. It should be understood that features and/or steps described with respect to one embodiment may be used with other embodiments and that not all embodiments of the invention have all of the features and/or steps shown in a particular figure or described with respect to one of the embodiments. Variations of embodiments described will occur to persons of the art. In particular, features described in the context of a method or process can be used to characterize a polymeric mixture and/or apparatus constructed therefrom. Similarly, features described in the context of a polymeric mixture and/or apparatus constructed therefrom can be used to characterize a method or process.

The terms “comprise,” “include,” “have” and their conjugates as used herein mean “including but not necessarily limited to”.

It is noted that some of the above described embodiments may describe the best mode contemplated by the inventors and therefore may include structure, acts or details of structures and acts that may not be essential to the invention and which are described as examples. Structure and acts described herein are replaceable by equivalents which perform the same function, even if the structure or acts are different, as known in the art. Therefore, the scope of the invention is limited only by the elements and limitations as used in the claims.

What is claimed is:

1. A medical probe, comprising:
a rigid proximal portion formed from a blend having a 1% secant flexural modulus of at least 1 GPa including at least 5% by weight of Liquid Crystal Polymer (LCP) having a melting point not lower than 280° C.;
a flexible distal portion having a shore A hardness lower than 90 A; and
a weld joining the distal portion to the proximal portion.

2. A probe according to claim 1, wherein the blend has a 1% secant flexural modulus of at least 1.2 GPa.

3. A probe according to claim 1, wherein the blend has a 1% secant flexural modulus of at least 1.5 GPa.

4. A probe according to claim 1, wherein the LCP has a 1% secant flexural modulus of at least 3 GPa.

5. A probe according to claim 1, wherein the LCP is a polyester.

6. A probe according to claim 1, wherein the rigid blend comprises a polymeric substance in addition to the LCP.

7. A probe according to claim 6, wherein the polymeric substance forms at least 30% of the blend.

8. A probe according to claim 6, wherein the polymeric substance comprises an engineering resin.

9. A probe according to claim 8, wherein the engineering resin has a modulus in the range of 1.0-2.0 GPa.

10. A probe according to claim 8, wherein the engineering resin has a modulus above 2.0 GPa.

11. A probe according to claim 10, wherein the engineering resin is selected from the group consisting of a polysulfone, a polyether ether ketone (PEEK), a polyphenylene sulfide (PPS), a polyphenylene ether (PPE), a polyphthalamide (PPA) and a polyimide (PI).

12. A probe according to claim 8, wherein the engineering resin comprises at least one of a polyester, a polyamide, a polyester copolymer and a polyamide copolymer.

13. A probe according to claim 12, wherein said polyamide is selected from the group consisting of polyamide 6, polyamide 66, polyamide 11, polyamide 12, polyamide 46, polyamide 6T, Polyphthalamide (PPA), blends and copolymers thereof.

14. A probe according to claim 12, wherein said polyester is selected from the group consisting of polyethylene terephthalate (PET), polybutylene terephthalate (PBT), glycol-modified polyethylene terephthalate (PETG), copolyesters and Poly ethylene naphthalate (PEN).

15. A probe according to claim 6, wherein the polymeric substance comprises a polyether block amide (PEBA).

16. A probe according to claim 6, comprising a compatibilizer forming at least 1% of the blend.

17. A probe according to claim 16, wherein the compatibilizer comprises at least 10% of the blend.

18. A probe according to claim 16, wherein the compatibilizer comprises a substantially greater portion of the blend than required for stable and repeatable coexistence of the LCP and the polymeric substance.

19. A probe according to claim 1, wherein the rigid blend further comprises at least 5% of a polymer or oligomer characterized by an average molecular weight not exceeding 20,000 Daltons.

20. A probe according to claim 19, wherein the polymer or oligomer has a melting point not exceeding 250° C.

21. A probe according to claim 19, wherein the polymer or oligomer has a softening point not exceeding 250° C.

22. A probe according to claim 1, wherein the LCP forms at least 20% of the blend.

23. A probe according to claim 1, wherein the blend forming the rigid proximal portion is weldable to thermoplastic polyurethane (TPU).

24. A probe according to claim 1, wherein the flexible distal portion comprises a TPU, selected from polyester-urethane, polyether-urethane, polycarbonate-urethane and silicone-urethane.

25. A probe according to claim 1, wherein the flexible distal portion has a shore A hardness lower than 75.

26. A probe according to claim 25, wherein the rigid proximal portion has a wall having a thickness of less than 0.2 millimeters.

27. A manufacturing method, comprising:
providing a first part formed from a rigid polymeric blend, comprising:
(i) at least 5% LCP having a melting point not lower than 280° C.; and
(ii) a polymeric substance,
wherein the blend is characterized by a 1% secant flexural modulus of at least 1.2 GPa;
further providing a second part formed from a flexible polymeric material; and
joining the first part and the second part by welding.

28. A method according to claim 27, wherein the flexible polymeric material comprises polyurethane.

29. A method according to claim 27, wherein the welding is performed by application of heat.

30. A method according to claim 27, wherein the welding is performed by application of a solvent.

31. A method according to claim 27, wherein each of the first and second parts are independently provided in the form of a rod or a tube.

32. A method according to claim 27, wherein the first part has a thickness of less than 0.2 millimeters.

33. A method according to claim 27, comprising adjusting a diameter of an end of at least one the first part and the second part so that the adjusted end fits over an end of the other part.

34. A method according to claim 33, wherein the adjusting includes at least one process selected from the group consisting of thermoforming, stretching and solvent swelling.

35. A method according to claim 27, wherein the flexible polymeric material has a shore hardness lower than 90 A at 20-25° C.
